(19) United States
(12) Patent Application Publication
(10) Pub. No.: US 2024/0325786 A1
(43) Pub. Date: Oct. 3, 2024

(54) COMBINING TUMOR TREATING FIELDS WITH RADIATION TREATMENT PLANNING

(71) Applicant: Novocure GmbH, Root D4 (CH)

(72) Inventors: Brian BERGER, New York, NY (US); Nadav SHAPIRA, Haifa (IL)

(73) Assignee: Novocure GmbH, Root D4 (CH)

(21) Appl. No.: 18/613,280

(22) Filed: Mar. 22, 2024

Related U.S. Application Data
(60) Provisional application No. 63/456,298, filed on Mar. 31, 2023.

(51) Int. Cl. A61N 5/10; A61N 1/40; G16H 20/40

(52) U.S. Cl. CPC A61N 5/1038; A61N 1/40; G16H 20/40

(57) ABSTRACT

A computer-implemented method comprising: obtaining a three-dimensional model of a subject; identifying a radiation therapy region in the model for delivering radiation therapy to a tumor of the subject, the radiation therapy region comprising a first and second radiation therapy regions to respectively receive a first and second dosage of radiation therapy; identifying a tumor treating fields therapy region in the model for delivering tumor treating fields therapy to the tumor, the tumor treating fields therapy region comprising the first radiation therapy region; identifying a first dosage for the tumor treating fields therapy for the first radiation therapy region to compensate for the first dosage of radiation therapy being smaller than the second dosage of radiation therapy; and selecting one or more transducer layouts for delivering tumor treating fields to the subject based on the first dosage for the tumor treating fields therapy for the first radiation therapy region.

### CROSS-REFERENCE TO RELATED APPLICATIONS

This application claims the benefit of and priority to U.S. Provisional Application No. 63/456,298, filed Mar. 31, 2023, the contents of which are incorporated herein by reference in its entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into the region of interest by transducers placed on the patient's body and applying alternating current (AC) voltages between the transducers. Conventionally, transducers used to generate TTFields include a plurality of electrode elements comprising ceramic disks. One side of each ceramic disk is positioned against the patient's skin, and the other side of each disc has a conductive backing. Electrical signals are applied to this conductive backing, and these signals are capacitively coupled into the patient's body through the ceramic discs. Conventional transducer designs include arrays of ceramic disks attached to a subject's body via a conductive skin-contact layer such as a hydrogel. AC voltage is applied between a pair of transducers for an interval of time to generate an electric field with field lines generally running in the front-back direction. Then, AC voltage is applied at the same frequency between at least another pair of transducers for another interval of time to generate an electric field with field lines generally running in the right-left direction. The system then repeats this two-step sequence throughout the treatment.

### BRIEF DESCRIPTION OF THE DRAWINGS

FIG. 1 depicts an example method for selecting one or more transducer layouts for delivering the TTFields to a subject.

FIGS. 2A-2D depict examples of various therapy regions.

FIG. 3 depicts an example apparatus to apply alternating electric fields to the subject's body.

FIG. 4 depicts an example system for attaching transducers to the subject's head for delivering TTFields.

FIGS. 5A and 5B depict example structures of a transducer array.

FIG. 6 depicts one example of an apparatus to determine the locations of transducers for applying TTFields to the head of the subject.

### DESCRIPTION OF EMBODIMENTS

This application describes exemplary techniques to computationally select and determine at least one transducer array layout for delivering TTFields to a subject.

Traditionally in tumor treatment, radiation is applied to treat where a tumor is identified and located. Radiation therapy usually causes severe side effects and may be difficult to apply. For example, the inherent toxicity of radiation treatment may cause side effects that may outweigh the benefits from treating a tumor. Further, the human body may also have a lifetime maximum limit to the effectiveness of radiation treatment. Moreover, it may be undesirable to apply radiation therapy to certain regions of a subject's body.

As an alternative or supplemental treatment, TTFields may be delivered to the subject, where TTFields may have much less side effects and have more flexibility in terms of tailoring tumor treatment plan. In general, in order to apply the TTFields to the subject's body, one or more pairs of transducers are positioned on the subject's body. Generally, at least two pairs of transducers are used. Transducers used to apply TTFields to the subject's body often include multiple electrode elements coupled together on a substrate.

The inventors discovered computational techniques to determine and select one or more transducer layouts for delivering the TTFields to a subject in addition to existing radiation therapy. The inventive techniques are particularly integrated into a practical application. For example, in some embodiments, treatment of more locations on the subject may be provided with less side effects compared to when using radiation alone. For example, in some embodiments, flexible deployment and combination of tumor treatment methods and dosage use may be employed for beneficial treatment of a tumor of the subject. In some embodiments, since the human body may have a lifetime maximum limit to the effectiveness of radiation treatment, a gap in treatment may occur, or may be prevented by using TTFields. As such, TTFields may be used to fill in the gap in coverage for radiation treatment by treating those areas of the subject that cannot receive radiation due to radiation-induced side effects and/or a lifetime maximum of radiation. In some embodiments, using TTFields may also simplify and/or speed up radiation treatment planning since a health care provider can use simpler radiation plans/procedures and supplementing with TTFields as needed.

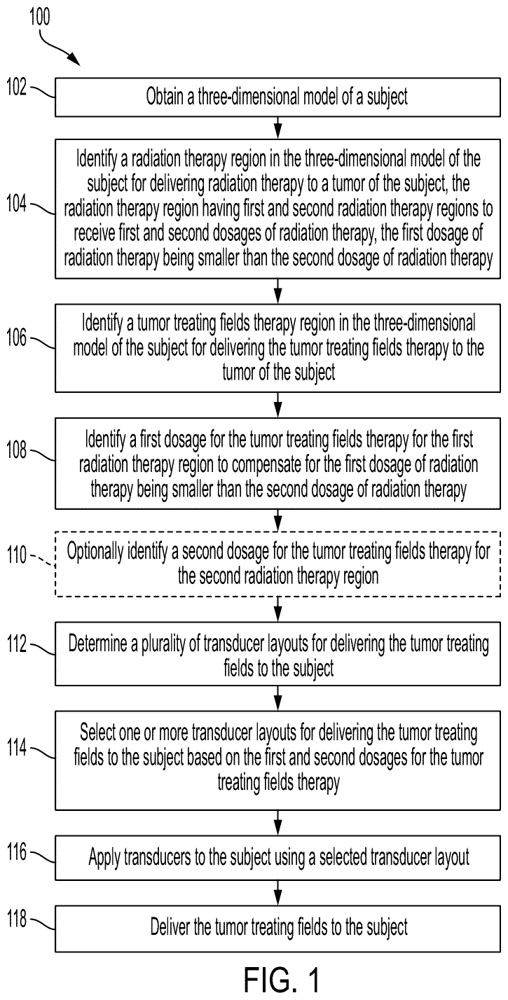

FIG. 1 depicts an example computer-implemented method 100 for selecting one or more transducer layouts for delivering the TTFields to a subject. The method 100 may be implemented by a computer, the computer including one or more processors and memory accessible by the one or more processors, the memory storing instructions that when executed by the one or more processors cause the computer to perform the steps of the method 100. Modifications, additions, or omissions may be made to method 100.

The method 100 includes, at Step 102, obtaining a three-dimensional (3D) model of the subject. The model comprises voxels. Each voxel of the model may be assigned a type of tissue (e.g., bone, organs, fluid, skin, or tumor) and/or an electrical conductivity associated with the type of tissue. As one example, the model of the subject may represent a head of the subject. As another example, the model of the subject may represent a torso of the subject. Other body parts of the subject may be represented in the model of the subject in other embodiments.

In some embodiments, the model may be obtained using image data, for example, via a computer identifying the different types of tissue from the image data. The image data may include one or more medical images of a portion of the subject's body (e.g., X-ray images, magnetic resonance imaging (MRI), computerized tomography (CT) images, ultrasound images, or any images providing an internal view of the subject's body). Each medical image may include an outer shape of a portion of the subject and a region corresponding to the region of interest (e.g., tumor) within the subject. The 3D model may be obtained, for example, from computer memory locally or over a network.

At Step 104, the method 100 may include identifying a radiation therapy region in the 3D model of the subject for delivering radiation therapy to a tumor of the subject. In some embodiments, the radiation therapy region may have a first radiation therapy region to receive a first dosage of radiation therapy and a second radiation therapy region to receive a second dosage of radiation therapy. In particular, the first radiation therapy region may be unable to safely absorb the second dosage of radiation therapy, for example a spinal region, optic chasm, optic tract, pituitary gland, brain stem, hypothalamus, parotid, bowel, or skin. In some embodiments, the first dosage of radiation therapy may be based on user input. In particular, the first and second dosages of radiation therapy may be in energy/mass units.

In some embodiments, the first radiation therapy region may correspond to a region of the subject that is more sensitive to radiation therapy than a region of the subject corresponding to the second radiation therapy region. In some embodiments, the first radiation therapy region may correspond to a spinal region of the subject. In some embodiments, the first radiation therapy region may correspond to a region of the subject that is more difficult to apply radiation therapy than a region of the subject corresponding to the second radiation therapy region.

In some embodiments, the first dosage of radiation therapy may be below a first threshold for the first radiation therapy region, wherein the first threshold is based on minimizing radiation therapy side effects for the subject and/or based on a limit of radiation therapy that may be applied to the first therapy region. In some embodiments, the first dosage of radiation therapy may be based on a region of the subject corresponding to the first radiation therapy region.

In some embodiments, the first dosage of radiation therapy may be up to 50% less than, or up to 50% greater than, the second dosage of radiation therapy, or any amount in between. In some embodiments, the first dosage of radiation therapy is 50% or less than and greater than 0% of the second dosage of radiation therapy. In some embodiments, the first dosage of radiation therapy is 20% or less than and greater than 0% of the second dosage of radiation therapy. As an example, a first dosage of radiation therapy may be 45 Gray (“Gy”) and the second dosage of radiation therapy may be 75 Gy. The first dosage of radiation therapy may be 35, 40, 45, 50, 55, 60, 65, 70, or 75 Gy, or any value therebetween. The region receiving the first dosage of radiation therapy may overlap, partially overlap, or not overlap the region receiving the second dosage of radiation therapy.

In some embodiments, the first and second dosages of radiation therapy may depend on the location of the tumor in the subject with respect to other tissue and/or organs of the subject. The dosages of radiation therapy may be limited by predetermined radiation tolerances for tissue and/or organs of the subject.

Examples of predetermined radiation tolerances for tissue and/or organs of a subject are discussed in, for example: Emami B., “Tolerance of Normal Tissue to Therapeutic Radiation,” Reports of Radiotherapy and Oncology, Spring 2013, Vol. 1, No. 1, pages 35-48 (hereinafter “Emami 2013”); Bisello S. et al., “Dose-Volume Constraints fOr oRganS At risk In Radiotherapy (CORSAIR): An “All-in-One” Multicenter-Multidisciplinary Practical Summary, “Current Oncology, 2022, 29, 7021-7050 (hereinafter “Bisello 2022”); Emami B. et al., “Tolerance of Normal Tissue to Therapeutic Irradiation,” Int J Radiat Oncol Biol Phys, 1991, 21:109-122 (hereinafter “Emami 1991”); and Marks L. B. et al., “Use of Normal Tissue Complication Probability Models in the Clinic,” Int. J. Radiation Oncology Biol. Phys., vol. 76, no. 3, Supplement, pp. S10-S19, 2010 (hereinafter “Marks 2010”), the contents of each of which are incorporated herein by reference in their entirety.

As examples, predetermined radiation tolerances for tissue and/or organs of a subject are discussed in, for example: Table 2 of Emami 2013, which is reproduced as Table 1 below; Table 1 of Bisello 2022; Table 1 of Emami 1991; and Table 1 of Marks 2010.

TABLE 1
Reproduced Table 2 of Emami 2013.

| | | Rate | Dose-volume | Dₘₐₓ | Dₘₑₐₙ |
|---|---|---|---|---|---|
| Organ | Endpoint | (%) | parameter | (Gy) | (Gy) |
| Brain | Symptomatic necrosis | <3 | | <60 | |
| | | <5 | | <65 | |
| Brainstem | Necrosis or cranial neuropathy | <5 | D100 < 54 Gy | | |
| | | <5 | D1-10 cc ≤ 59 Gy | <64 Point | |
| Spinal cord | Grade ≥2 myelopathy | <1 | | 50 | |
| Optic nerve & | Optic neuropathy | <3 | | <55 | <50 |
| chiasm | | 3-7 | | 55-60 | |
| Retina | Blindness | <1 | | <50 | |
| Cochlea | Hearing loss | <15 | | | ≤45 |
| Parotid 1 | Grade 4 xerostomia | <20 | | | <20 |
| Parotid 2 | | <20 | | | <25 |
| Mandible | ORN | <5 | | <70 Point | |
| Pharyngeal | PEG tube dependent | <5 | | | <50 |
| constrictors | Aspiration | <5 | | | <60 |
| Larynx | Grade ≥2 edema | <20 | V50 < 27% | | <44 |
| Brachial | Clinically apparent nerve | <5 | | <60 | |
| plexus | damage | | | | |
| Lung | Symptomatic pneumonitis | 5 | V5 < 42%, | | 7 |
| | | | V20 < 22% | | |
| | | 10 | V20 < 31% | | 13 |
| | | 20 | V20 < 40% | | 20 |
| | | 30 | | | 24 |
| | | 40 | | | 27 |
| Esophagus | Grade ≥2 esophagitis | <30 | V35 < 50% | <74 Point | |
| | | | V50 < 40% | | |
| | | | V70 < 20% | | |
| | Grade ≥3 esophagitis | ≤10 | V60 < 30% | | <34 |
| Heart | Pericarditis | <15 | V30 < 46% | | <26 |
| | Long-term cardiac mortality | <1 | V25 < 10% | | |
| Liver | RILD, normal liver | <5 | | | ≤30 |
| | RILD, liver disease | <5 | | | ≤28 |
| Kidney 1 | Renal dysfunction | <5 | Equivalent of 1 | | |
| | | | kidney <18 Gy | | |
| Kidney 2 | Renal dysfunction | <5 | | | <18 |
| Stomach | Ulceration | | D100 < 50 Gy | | |
| Small Bowel | Acute grade ≥3 toxicity | <10 | V15 < 120 cc | | |
| | Late obstruction/perforation | <5 | V50 < 5% | | |
| Rectum | Grade ≥2/≥3 late toxicity | <10/<15 | V50 < 50% | | |
| | Grade ≥2/≥3 late toxicity | <10/<15 | V60 < 35% | | |
| | Grade ≥2/≥3 late toxicity | <10/<15 | V65 < 25% | | |
| | Grade ≥2/≥3 late toxicity | <10/<15 | V70 < 20% | | |
| | Grade ≥2/≥3 late toxicity | <10/<15 | V75 < 15% | | |
| Bladder | Grade ≥3 late toxicity | <6 | D100 < 65 Gy | | |
| | | ? | V65 ≤ 50% | | |
| | | | V70 ≤ 35% | | |
| | | | V75 ≤ 25% | | |
| | | | V80 ≤ 15% | | |
| Penile bulb | Severe erectile dysfunction | <35 | | | <50 |
| Femoral head | Necrosis | <5 | D100 < 52 Gy | | |

Parotid 1, sparing single parotid gland; Parotid 2, combined parotid glands; Kidney 1, bilateral partial kidney RT; Kidney 2, bilateral whole kidneys; Vx, volume of the organ receiving ≥x Gy; Dx, minimum dose received by x% of the organ; Dₘₐₓ maximum radiation dose; Dₘₑₐₙ mean radiation dose.

Use of the terms “first” and “second” as discussed herein with regards to dosages designate separate dosage amounts and do not necessarily indicate an order of application of the dosages. In some embodiments, the first dosage of radiation therapy may be applied in one, two, or more portions at separate times to obtain the first dosage of radiation therapy. In some embodiments, the second dosage of radiation therapy may be applied in one, two, or more portions at separate times to obtain the second dosage of radiation therapy. In some embodiments, a portion of the first dosage of radiation therapy and a portion of the second dosage of radiation therapy may be applied at the same time or at different times to the subject.

In some embodiments, more than two radiation therapy regions may be needed. The number of radiation therapy regions may depend on the number of tumor locations and/or their locations with respect to tissue and/or organs of the subject.

At Step 106, the method 100 may include identifying a TTFields therapy region in the 3D model of the subject for delivering the TTFields therapy to the tumor of the subject. In some embodiments, the TTFields therapy region may at least partially include the first radiation therapy region. In some embodiments, the TTFields therapy region may at least partially include the second radiation therapy region.

At Step 108, the method 100 may include identifying a first dosage for the TTFields therapy for the first radiation therapy region to compliment the first dosage of radiation therapy to achieve a cumulative dosage. The cumulative dosage may be a pre-determined therapeutic amount of dosage sufficient to treat or remove a tumor or cancerous tissue. In some embodiments, the cumulative dosage may be substantially equal to or greater than the second radiation dosage. In some embodiments, the first dosage for the tumor treating fields therapy for the first radiation therapy region may be based on the first dosage of radiation therapy for the first radiation therapy region. In particular, the first dosage for the TTFields therapy may be in energy/volume units.

More specifically, identifying the first dosage for the tumor treating fields therapy for the first radiation therapy region may include determining an approximate equivalent first dosage of radiation therapy for the first radiation therapy region in energy/volume units, and determining the first dosage for the tumor treating fields therapy for the first radiation therapy region based on the approximate equivalent first dosage of radiation therapy for the first radiation therapy region.

In some embodiments, a combination (cumulative dosage) of the first dosage for the TTFields therapy for the first radiation therapy region and the first dosage of radiation therapy for the first radiation therapy region may meet or exceed a therapeutic dosage threshold for treating the tumor in the first radiation therapy region using radiation therapy alone. In some embodiments, the first dosage of radiation therapy for the first radiation therapy region may be less than a therapeutic dosage threshold for treating the tumor in the first radiation therapy region using radiation therapy alone. In some embodiments, a combination (cumulative dosage) of the first dosage for the TTFields therapy for the first radiation therapy region and the first dosage of radiation therapy for the first radiation therapy region may be less than a therapeutic dosage threshold for treating the tumor in the first radiation therapy region using the radiation therapy alone. In some embodiments, a combination (cumulative dosage) of the first dosage for the TTFields therapy for the first radiation therapy region and the first dosage of radiation therapy for the first radiation therapy region may be approximately equivalent to the second dosage of radiation therapy.

Different organs or areas of the subject have various therapeutic dosage thresholds for treating a tumor using radiation therapy alone. For example, the bowel area of a subject may have a therapeutic dosage threshold between 45-50 Gy, and the spinal area of the subject may have a therapeutic dosage threshold between 45-54 Gy. As one example, when applying radiation therapy to a region of the pancreas, a therapeutic dosage threshold for treating a tumor in a radiation therapy region may be 45 Gy, because a higher dosage may perforate the bowel of the subject located next to the pancreas. In such an example, in treating the tumor in the region of pancreas, a dosage for the TTFields therapy for the first radiation therapy region may be applied in addition to a dosage of 45 Gy of radiation therapy for the first radiation therapy region, resulting a combination of dosage exceeding a therapeutic dosage threshold of using radiation therapy alone.

At optional Step 110, the method 100 may include identifying a second dosage for the TTFields therapy for the second radiation therapy region based on the second dosage of radiation therapy for the second radiation therapy region. However, in some embodiments, the method may include identifying a second dosage for the tumor treating fields therapy for the second radiation therapy region without regard to the second dosage of radiation therapy for the second radiation therapy region.

In some embodiments, more than two dosages of tumor treating therapy may be needed. The number of tumor treating therapies may depend on the number of tumor locations, the radiation dosages, the radiation therapy regions, and/or locations of the radiation therapy regions with respect to tissue and/or organs of the subject.

At Step 112, the method 100 may include determining a plurality of transducer layouts for delivering TTFields to the subject. In some embodiments, the transducer layouts in the plurality of transducer layouts may differ by at least one of location on the subject, size of the transducer, shape of the transducer, number of electrodes of the transducer, size of electrodes of the transducer, shape of electrodes of the transducer, or applied material and/or structure of the transducer.

At Step 114, the method 100 may include selecting one or more transducer layouts from the plurality of transducer layouts from the plurality of transducer layouts for delivering the TTFields to the subject. In some embodiments, the selection may be based on the first dosage for the TTFields therapy for the first radiation therapy region. In some embodiments, the selection may be further based on the second dosage for the tumor treating fields therapy for the second radiation therapy region.

In some embodiments, at least one of the selected transducer layouts may be capable of providing the first dosage for the tumor treating fields therapy for the first radiation therapy region and a second dosage of the tumor treating fields therapy for the second radiation therapy region. In some embodiments, at least one of the selected transducer layouts may be capable of providing the first dosage for the tumor treating fields therapy for the first radiation therapy region and capable of providing a different dosage for the tumor treating fields therapy to at least one other region of the subject. In particular, the first dosage of the tumor treating fields therapy and the second dosage of the tumor treating fields therapy may be different.

At Step 116, the method 100 may include applying transducers to the subject using the selected transducer layout.

At Step 118, the method 100 may include delivering the TTFields to the subject.

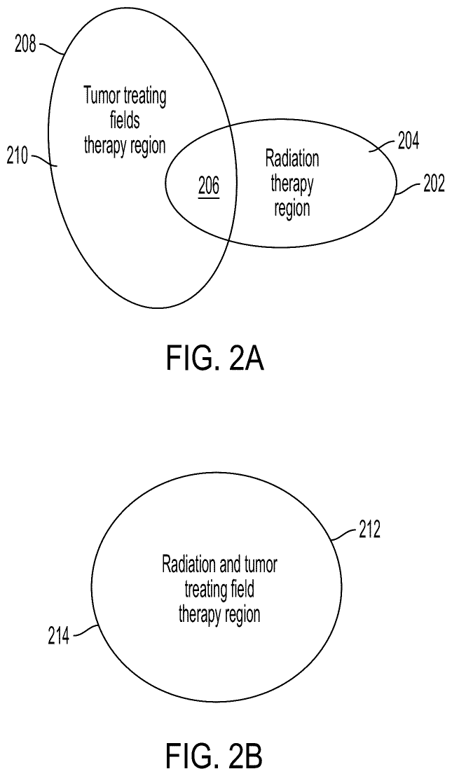

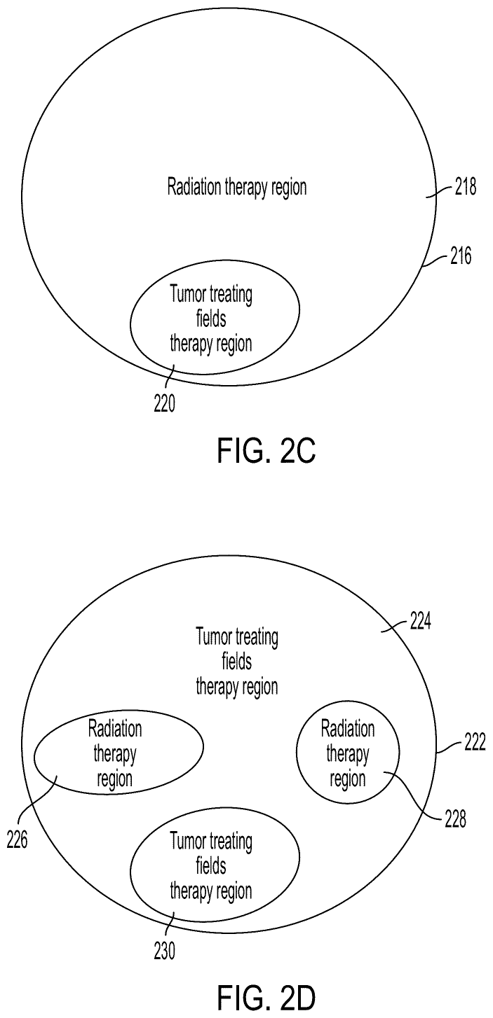

FIGS. 2A-2D depict examples of various therapy regions. For example, FIG. 2A shows a radiation therapy region 202 having therapy regions 204 and 206 and a TTFields therapy region 208 having therapy regions 206 and 210. Therapy regions 204 and 206 may receive different radiation therapies, and therapy regions 206 and 210 may receive different TTFields therapies. Therapy region 204 may only receive radiation therapy, and may correspond to a section of a tumor away from a sensitive or hard to reach area. Therapy region 206 may receive both radiation therapy and TTFields therapy, and may correspond to a section of a tumor within or adjacent to a sensitive or hard to reach area. Therapy region 210 may only receive TTFields therapy.

FIG. 2B shows a radiation therapy region 212 that includes a full overlap with a TTFields therapy region 214. Such a region may correspond to a tumor fully situated within or adjacent to a sensitive or hard to reach tissue.

FIG. 2C shows a radiation therapy region 216 having a therapy region 218 that undergoes radiation alone, and may correspond to a section of a tumor away from a sensitive or hard to reach area. Within the radiation therapy region 216 is a region 220 that receives both radiation and TTField therapies. Region 220 may correspond to a section of a tumor within or adjacent to a sensitive or hard to reach area.

FIG. 2D shows a TTFields therapy region 222 having a therapy region 224 that undergoes TTFields therapy alone, and may correspond to a section of a tumor within or adjacent to a sensitive or hard to reach area and/or a section including potential locations for a tumor. Within the TTFields therapy region 222 are three radiation therapy regions 226, 228, and 230 that receive both radiation and TTFields therapies. Regions 226, 228, and 230 may correspond to a section of a tumor away from sensitive or hard to reach area. In some embodiments, the number of the radiation therapy regions may not limited to three and may be one, two, four, five, or more.

Geographically, a tumor may be adjacent to normal tissue and/or may abut one or more organs of the subject. The interplay between the TTFields therapy and the radiation therapy may effectively enable a reduction in radiation dosage when applying treatment to the subject. In some embodiments, FIG. 2A may represent applying treatment to a tumor adjacent an organ, for example, a tumor adjacent the bowel of a subject. For example, the TTFields therapy in region 208 may increase or compensate therapeutic dosage applied to the therapy region 206 only, so that the radiation dosage applied to the therapy region 206 may be reduced and/or does not exceed the corresponding therapeutic dosage threshold using radiation therapy alone.

In some embodiments, FIG. 2B may represent applying treatment to a tumor in the middle of an organ or area, such as a tumor in spine area of the subject. For example, the TTFields therapy in region 214 may increase or compensate the therapeutic dosage applied to the entire therapy region 212, so that the radiation dosage applied to the therapy region 212 may be reduced and/or may not exceed the corresponding therapeutic dosage threshold using radiation therapy alone.

In some embodiments, FIG. 2B may also represent applying treatment to a tumor in an organ that limits radiation dosage. For example, a lung (or both lungs) of the subject may have a limit of receiving radiation dosage based on size of the lung. When applying radiation therapy to the lung, radiation dosage may need to be applied to the entire lung area at a certain minimum level while higher dosage is required for the tumor area. This may cause the entire radiation dosage for the lung to exceed the limit of receiving radiation dosage. In such example, the TTFields therapy may be applied to the entire therapy region 212 to compensate for the radiation dosage applied to the entire lung area, so that the entire radiation dosage does not exceed the radiation limit.

In some embodiments, FIG. 2C may represent another example of applying treatment to a tumor in an organ that limits radiation dosage. For example, in addition to applying the radiation therapy to the radiation therapy region 218, such as a lung area of the subject, the TTFields therapy may be applied to the TTFields therapy region 220 to compensate for the therapeutic dosage applied to the tumor, so that the entire radiation dosage does not exceed the radiation limit.

In some embodiments, FIG. 2C may also represent applying treatment to the tumor about an organ of the subject. For example, in applying radiation therapy to the radiation therapy region 218, such as adjacent to and/or within the bowel of the subject, the TTFields therapy may be applied to the TTFields therapy region 220 only within the bowel area, so that the region 220 may receive additional therapeutic dosage.

In some embodiments, FIG. 2D may represent applying treatment when not all of the tumor locations are detectable, for example, a spindly tumor in the brain of a subject. For example, the whole brain of the subject may include more than one tumor location, and there may be concern that not all locations of the tumor were detected in the medical image. In such a situation, the whole brain may need to receive radiation treatment; however, an entire necessary dosage of radiation therapy may cause neuro toxicity. For example, tolerance radiation dosage of the whole brain may be 60 Gy, while a minimum dosage of 60 Gy may be needed to treat each individual tumor location. In such an example, radiation therapy may be applied to local areas 226, 228, and 230, which are determined to be the tumor locations, with lower dosage, such as 10 Gy for each local area 226, 228, and 230. In addition, the TTFields therapy may be applied to the TTFields therapy region 224, such as the whole brain, to compensate the therapeutic dosage to the whole brain, including local areas 226, 228, and 230 determined to have the tumor and areas of the brain where no tumor is yet detected.

### Exemplary Apparatuses

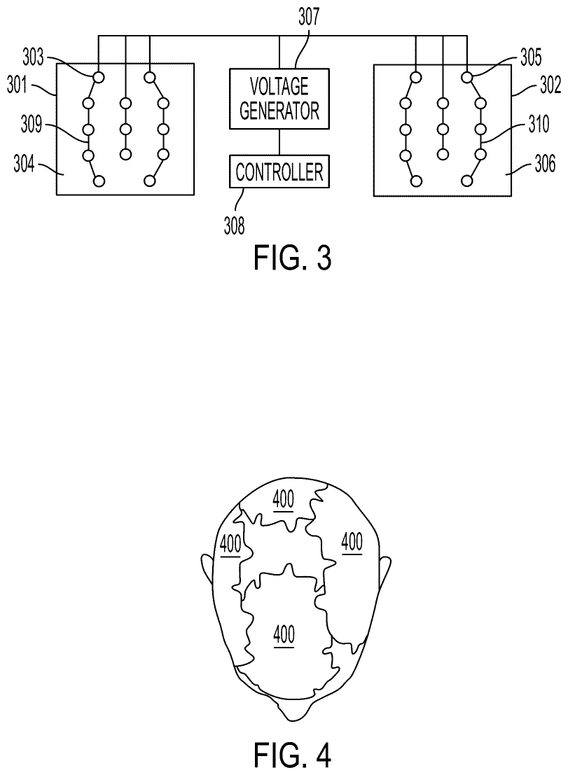

FIG. 3 depicts an example apparatus to apply alternating electric fields (e.g., TTFields) to the subject's body. The first transducer array 301 includes thirteen electrode elements 303, which are positioned on the substrate 304, and the electrode elements 303 are electrically and mechanically connected to one another through a conductive wiring 309. The second transducer array 302 includes thirteen electrode elements 305, which are positioned on the substrate 306, and the electrode elements 305 are electrically and mechanically connected to one another through a conductive wiring 310. The first transducer array 301 and the second transducer array 302 are connected to an AC voltage generator 307 and a controller 308. The controller 308 may include one or more processors and memory accessible by the one or more processors. The memory may store instructions that when executed by the one or more processors, control the AC voltage generator 307 to implement one or more embodiments of the invention. In some embodiments, the AC voltage generator 307 and the controller 308 may be integrated in the first transducer array 301 and the second transducer array 302 and form a first electric field generator and a second electric field generator.

The structure of the transducers may take many forms. The transducers may be affixed to the subject's body or attached to or incorporated in clothing covering the subject's body. The transducer may include suitable materials for attaching the transducer to the subject's body. For example, the suitable materials may include cloth, foam, flexible plastic, and/or a conductive medical gel. The transducer may be conductive or non-conductive. In some embodiments, a target region may be in the subject's brain, and the TTFields may be delivered to the subject's head via the two pairs of transducer arrays positioned on a head of the subject's body (such as, for example, as shown in FIG. 4, which has four transducer arrays 400). As another example, the target region may be in the subject's lung, and the TTFields may be delivered to the subject's body via two pairs of transducer arrays positioned around chest and back of the subject's body.

The transducer may include any desired number of electrode elements. Various shapes, sizes, and materials may be used for the electrode elements. Any constructions for implementing the transducer (or electric field generating device) for use with embodiments of the invention may be used as long as they are capable of (a) delivering TTFields to the subject's body and (b) being positioned at the locations specified herein. In certain embodiments, at least one electrode element of the first, the second, the third, or the fourth transducer can include at least one ceramic disk that is adapted to generate an alternating electric field. In non-limiting embodiments, at least one electrode element of the first, the second, the third, or the fourth transducer comprises a polymer film that is adapted to generate an alternating field.

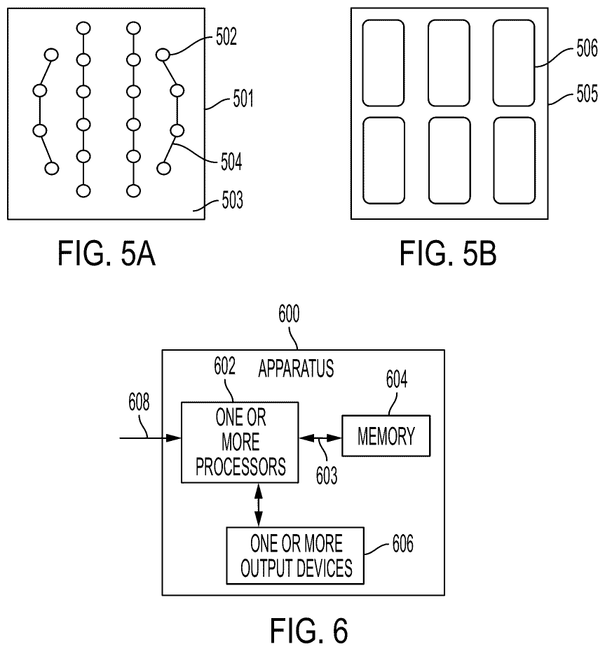

FIG. 5A depicts one example of an alternative design of the transducer array. The transducer array 501 includes twenty electrode elements 502, which are positioned on the substrate 503, and the electrode element 502 are electrically and mechanically connected to one another through a conductive wiring 504. In some embodiments, the electrode elements 502 can include a ceramic disk.

FIG. 5B depicts one example of an alternative design of the transducer array. The transducer 505 can include substantially flat electrode elements 506. In some embodiments, the electrode elements 506 are non-ceramic dielectric materials positioned over a plurality of flat conductors. Examples of non-ceramic dielectric materials positioned over flat conductors may include polymer films disposed over pads on a printed circuit board or over flat pieces of metal. In other embodiments, the electrode elements 506 are ceramic elements. In non-limiting embodiments, the electrode elements 502 and 506 can have various shapes. For example, the electrode elements can be triangular, rectangular, circular, oval, ovaloid, ovoid, or elliptical in shape or substantially triangular, substantially rectangular, substantially circular, substantially oval, substantially ovaloid, substantially ovoid, or substantially elliptical in shape.

FIG. 6 depicts an example computer apparatus for use with the embodiments herein. As an example, the apparatus 600 may be a computer to implement certain inventive techniques disclosed herein, such as selecting at least one transducer layout for delivering the TTFields to a subject according to FIG. 1. For example, Steps 102 to 114 of FIG. 1 may be performed by a computer, such as computer apparatus 600. As an example, the apparatus 600 may be used as the controller 308 of FIG. 3, or as a separate computer apparatus located remote from the controller 308. For example, Step 118 of FIG. 1 may be performed by a controller, such as controller 308. The apparatus 600 may include one or more processors 602, a memory 604, and one or more output devices 606.

In one example, based on input 608, the one or more processors 602 generate control signals to control the voltage generator. As one example, the input 608 is user input from one or more input device(s) (not shown). As another example, the input 608 may be from another computer in communication with the controller apparatus 600. The memory 604 is accessible by the one or more processors 602 (e.g., via a link 603) so that the one or more processors 602 can read information from and write information to the memory 604. The memory 604 may store instructions that when executed by the one or more processors 602 implement one or more methods of the present disclosure. The one or more output devices 606 may provide the status of the operation of the invention, such as transducer layout selection, voltages being generated, and other operational information. The output device(s) 606 may provide visualization data according to certain embodiments of the invention.

### ILLUSTRATIVE EMBODIMENTS

The invention includes other illustrative embodiments (“Embodiments”) as follows.
Embodiment 1: A computer-implemented method for selecting at least one transducer layout for delivering tumor treating fields to a subject, the method comprising: obtaining a three-dimensional model of the subject, the model comprising voxels; identifying a radiation therapy region in the three-dimensional model of the subject for delivering radiation therapy to a tumor of the subject, the radiation therapy region comprising a first radiation therapy region to receive a first dosage of radiation therapy and a second radiation therapy region to receive a second dosage of radiation therapy, the first dosage of radiation therapy being smaller than the second dosage of radiation therapy; identifying a tumor treating fields therapy region in the three-dimensional model of the subject for delivering tumor treating fields therapy to the tumor of the subject, the tumor treating fields therapy region comprising the first radiation therapy region; identifying a first dosage for the tumor treating fields therapy for the first radiation therapy region to compensate for the first dosage of radiation therapy being smaller than the second dosage of radiation therapy; and selecting one or more transducer layouts for delivering tumor treating fields to the subject based on the first dosage for the tumor treating fields therapy for the first radiation therapy region.
Embodiment 2: The computer-implemented method of claim 1, wherein the first radiation therapy region corresponds to a region of the subject that is more sensitive to radiation therapy than a region of the subject corresponding to the second radiation therapy region.
Embodiment 3: The computer-implemented method of claim 2, wherein the first radiation therapy region corresponds to a spinal region of the subject.
Embodiment 4: The computer-implemented method of claim 1, wherein the first dosage of radiation therapy is below a first threshold for the first radiation therapy region, wherein the first threshold is based on minimizing radiation therapy side effects for the subject.
Embodiment 5: The computer-implemented method of claim 1, wherein the first radiation therapy region corresponds to a region of the subject that is more difficult to apply radiation therapy than a region of the subject corresponding to the second radiation therapy region.
Embodiment 6: The computer-implemented method of claim 4, wherein the first radiation therapy region corresponds to a brain or lung region of the subject.
Embodiment 7: The computer-implemented method of claim 1, wherein the first radiation therapy region is unable to safely absorb the second dosage of radiation therapy.
Embodiment 8: The computer-implemented method of claim 1, wherein the first dosage of radiation therapy is based on a region of the subject corresponding to the first radiation therapy region.
Embodiment 9: The computer-implemented method of claim 1, wherein the first dosage of radiation therapy is 50% or less than and greater than 0% of the second dosage of radiation therapy.
Embodiment 10: The computer-implemented method of claim 1, wherein the first dosage of radiation therapy is 20% or less than and greater than 0% of the second dosage of radiation therapy.
Embodiment 11: The computer-implemented method of claim 1, wherein the first dosage of radiation therapy is based on user input.
Embodiment 12: The computer-implemented method of claim 1, wherein the first dosage for the tumor treating fields therapy for the first radiation therapy region is based on the first dosage of radiation therapy for the first radiation therapy region.
Embodiment 13: The computer-implemented method of claim 1, wherein the tumor treating fields therapy region in the three-dimensional model of the subject comprises a portion or all of the second radiation therapy region, wherein the method further comprises identifying a second dosage for the tumor treating fields therapy for the second radiation therapy region based on the second dosage of radiation therapy for the second radiation therapy region, and wherein selecting one or more transducer layouts for delivering tumor treating fields to the subject is further based on the second dosage for the tumor treating fields therapy for the second radiation therapy region.
Embodiment 14: The computer-implemented method of claim 1, wherein the tumor treating fields therapy region in the three-dimensional model of the subject comprises a portion or all of the second radiation therapy region, wherein the method further comprises identifying a second dosage for the tumor treating fields therapy for the second radiation therapy region without regard to the second dosage of radiation therapy for the second radiation therapy region, and wherein selecting one or more transducer layouts for delivering tumor treating fields to the subject is further based on the second dosage for the tumor treating fields therapy for the second radiation therapy region.
Embodiment 15: The computer-implemented method of claim 1, wherein a combination of the first dosage for the tumor treating fields therapy for the first radiation therapy region and the first dosage of radiation therapy for the first radiation therapy region meets or exceeds a therapeutic dosage threshold for treating the tumor in the first radiation therapy region using radiation therapy alone, wherein the first dosage of radiation therapy for the first radiation therapy region is less than a therapeutic dosage threshold for treating the tumor in the first radiation therapy region using radiation therapy alone.
Embodiment 16: The computer-implemented method of claim 1, wherein a combination of the first dosage for the tumor treating fields therapy for the first radiation therapy region and the first dosage of radiation therapy for the first radiation therapy region is less than a therapeutic dosage threshold for treating the tumor in the first radiation therapy region using the radiation therapy alone.
Embodiment 17: The computer-implemented method of claim 1, wherein the first dosage of radiation therapy is in energy/mass units, wherein the first dosage for the tumor treating fields therapy is in energy/volume units, and wherein identifying the first dosage for the tumor treating fields therapy for the first radiation therapy region comprises: determining an approximate equivalent first dosage of radiation therapy for the first radiation therapy region in energy/volume units; and determining the first dosage for the tumor treating fields therapy for the first radiation therapy region based on the approximate equivalent first dosage of radiation therapy for the first radiation therapy region.
Embodiment 18: The computer-implemented method of claim 1, wherein the first and second dosages of radiation therapy are in energy/mass units, wherein a cumulative dosage of the first dosage for the tumor treating fields therapy for the first radiation therapy region and the first dosage of radiation therapy for the first radiation therapy region is approximately equivalent to or greater than the second dosage of radiation therapy.
Embodiment 19: The computer-implemented method of claim 1, wherein at least one of the selected transducer layouts is capable of providing the first dosage for the tumor treating fields therapy for the first radiation therapy region and a second dosage of the tumor treating fields therapy for the second radiation therapy region, wherein the first dosage of the tumor treating fields therapy and the second dosage of the tumor treating fields therapy are different.
Embodiment 20: The computer-implemented method of claim 1, wherein at least one of the selected transducer layouts is capable of providing the first dosage for the tumor treating fields therapy for the first radiation therapy region and capable of providing a different dosage for the tumor treating fields therapy to at least one other region of the subject.
Embodiment 21: The computer-implemented method of claim 1, further comprising determining a plurality of transducer layouts for delivering tumor treating fields to the subject, wherein the at least one transducer layout is selected from the plurality of transducer layouts, wherein the transducer layouts in the plurality of transducer layouts differ by at least one of location on the subject, size of the transducer, shape of the transducer, number of electrodes of the transducer, size of electrodes of the transducer, or shape of electrodes of the transducer.
Embodiment 22: An apparatus for selecting at least one transducer layout for delivering tumor treating fields to a subject, the apparatus comprising: one or more processors; and memory accessible by the one or more processors, the memory storing instructions that when executed by the one or more processors, cause the apparatus to: obtain a three-dimensional model of the subject, the model comprising voxels; identify a radiation therapy region in the three-dimensional model of the subject for delivering radiation therapy to a tumor of the subject, the radiation therapy region comprising a first radiation therapy region to receive a first dosage of radiation therapy, the first dosage of radiation therapy being smaller than a therapeutic threshold for treating the tumor in the first radiation therapy region; identify a tumor treating fields therapy region in the three-dimensional model of the subject for delivering tumor treating fields therapy to the tumor of the subject, the tumor treating fields therapy region comprising the first radiation therapy region; identify a first dosage for the tumor treating fields therapy for the first radiation therapy region, wherein combining the first dosage of radiation therapy and the first dosage for the tumor treating fields therapy in the first radiation therapy region meets or exceed a therapeutic threshold for treating the tumor in the first radiation therapy region; and select one or more transducer layouts for delivering tumor treating fields to the subject based on the first dosage for the tumor treating fields therapy for the first radiation therapy region.
Embodiment 23: A non-transitory processor readable medium containing a set of instructions thereon that when executed by a processor cause the processor to: obtain a three-dimensional model of the subject, the model comprising voxels; identify a radiation therapy region in the three-dimensional model of the subject for delivering radiation therapy to a tumor of the subject, the radiation therapy region comprising a first radiation therapy region to receive a first dosage of radiation therapy and a second radiation therapy region to receive a second dosage of radiation therapy, the first dosage of radiation therapy being less than the second dosage of radiation therapy; identify a tumor treating fields therapy region in the three-dimensional model of the subject for delivering tumor treating fields therapy to the tumor of the subject, the tumor treating fields therapy region comprising the first radiation therapy region; identify a first dosage for the tumor treating fields therapy for the first radiation therapy region based on the first dosage of radiation therapy for the first radiation therapy region; and select one or more transducer layouts for delivering tumor treating fields to the subject based on the first dosage for the tumor treating fields therapy for the second radiation therapy region

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A computer-implemented method for selecting at least one transducer layout for delivering tumor treating fields to a subject, the method comprising:
obtaining a three-dimensional model of the subject, the model comprising voxels;
identifying a radiation therapy region in the three-dimensional model of the subject for delivering radiation therapy to a tumor of the subject, the radiation therapy region comprising a first radiation therapy region to receive a first dosage of radiation therapy and a second radiation therapy region to receive a second dosage of radiation therapy, the first dosage of radiation therapy being smaller than the second dosage of radiation therapy;
identifying a tumor treating fields therapy region in the three-dimensional model of the subject for delivering tumor treating fields therapy to the tumor of the subject, the tumor treating fields therapy region comprising the first radiation therapy region;
identifying a first dosage for the tumor treating fields therapy for the first radiation therapy region to compensate for the first dosage of radiation therapy being smaller than the second dosage of radiation therapy; and
selecting one or more transducer layouts for delivering tumor treating fields to the subject based on the first dosage for the tumor treating fields therapy for the first radiation therapy region.

2. The computer-implemented method of claim 1, wherein the first radiation therapy region corresponds to a region of the subject that is more sensitive to radiation therapy than a region of the subject corresponding to the second radiation therapy region.

3. The computer-implemented method of claim 2, wherein the first radiation therapy region corresponds to a spinal region of the subject.

4. The computer-implemented method of claim 1, wherein the first dosage of radiation therapy is below a first threshold for the first radiation therapy region, wherein the first threshold is based on minimizing radiation therapy side effects for the subject.

5. The computer-implemented method of claim 1, wherein the first radiation therapy region corresponds to a region of the subject that is more difficult to apply radiation therapy than a region of the subject corresponding to the second radiation therapy region.

6. The computer-implemented method of claim 4, wherein the first radiation therapy region corresponds to a brain or lung region of the subject.

7. The computer-implemented method of claim 1, wherein the first radiation therapy region is unable to safely absorb the second dosage of radiation therapy.

8. The computer-implemented method of claim 1, wherein the first dosage of radiation therapy is based on a region of the subject corresponding to the first radiation therapy region.

9. The computer-implemented method of claim 1, wherein the first dosage of radiation therapy is 50% or less than and greater than 0% of the second dosage of radiation therapy.

10. The computer-implemented method of claim 1, wherein the first dosage of radiation therapy is based on user input.

11. The computer-implemented method of claim 1, wherein the first dosage for the tumor treating fields therapy for the first radiation therapy region is based on the first dosage of radiation therapy for the first radiation therapy region.

12. The computer-implemented method of claim 1, wherein the tumor treating fields therapy region in the three-dimensional model of the subject comprises a portion or all of the second radiation therapy region,
wherein the method further comprises identifying a second dosage for the tumor treating fields therapy for the second radiation therapy region based on the second dosage of radiation therapy for the second radiation therapy region, and
wherein selecting one or more transducer layouts for delivering tumor treating fields to the subject is further based on the second dosage for the tumor treating fields therapy for the second radiation therapy region.

13. The computer-implemented method of claim 1, wherein the tumor treating fields therapy region in the three-dimensional model of the subject comprises a portion or all the second radiation therapy region,
wherein the method further comprises identifying a second dosage for the tumor treating fields therapy for the second radiation therapy region without regard to the second dosage of radiation therapy for the second radiation therapy region, and
wherein selecting one or more transducer layouts for delivering tumor treating fields to the subject is further based on the second dosage for the tumor treating fields therapy for the second radiation therapy region.

14. The computer-implemented method of claim 1, wherein a combination of the first dosage for the tumor treating fields therapy for the first radiation therapy region and the first dosage of radiation therapy for the first radiation therapy region meets or exceeds a therapeutic dosage threshold for treating the tumor in the first radiation therapy region using radiation therapy alone,
wherein the first dosage of radiation therapy for the first radiation therapy region is less than a therapeutic dosage threshold for treating the tumor in the first radiation therapy region using radiation therapy alone.

15. The computer-implemented method of claim 1, wherein the first dosage of radiation therapy is in energy/mass units,
wherein the first dosage for the tumor treating fields therapy is in energy/volume units, and
wherein identifying the first dosage for the tumor treating fields therapy for the first radiation therapy region comprises:
determining an approximate equivalent first dosage of radiation therapy for the first radiation therapy region in energy/volume units; and
determining the first dosage for the tumor treating fields therapy for the first radiation therapy region based on the approximate equivalent first dosage of radiation therapy for the first radiation therapy region.

16. The computer-implemented method of claim 1, wherein the first and second dosages of radiation therapy are in energy/mass units,
wherein a cumulative dosage of the first dosage for the tumor treating fields therapy for the first radiation therapy region and the first dosage of radiation therapy for the first radiation therapy region is approximately equivalent to or greater than the second dosage of radiation therapy.

17. The computer-implemented method of claim 1, wherein at least one of the selected transducer layouts is capable of providing the first dosage for the tumor treating fields therapy for the first radiation therapy region and a second dosage of the tumor treating fields therapy for the second radiation therapy region,
wherein the first dosage of the tumor treating fields therapy and the second dosage of the tumor treating fields therapy are different.

18. The computer-implemented method of claim 1, wherein at least one of the selected transducer layouts is capable of providing the first dosage for the tumor treating fields therapy for the first radiation therapy region and capable of providing a different dosage for the tumor treating fields therapy to at least one other region of the subject.

19. An apparatus for selecting at least one transducer layout for delivering tumor treating fields to a subject, the apparatus comprising: one or more processors; and memory accessible by the one or more processors, the memory storing instructions that when executed by the one or more processors, cause the apparatus to:
obtain a three-dimensional model of the subject, the model comprising voxels;
identify a radiation therapy region in the three-dimensional model of the subject for delivering radiation therapy to a tumor of the subject, the radiation therapy region comprising a first radiation therapy region to receive a first dosage of radiation therapy, the first dosage of radiation therapy being smaller than a therapeutic threshold for treating the tumor in the first radiation therapy region;
identify a tumor treating fields therapy region in the three-dimensional model of the subject for delivering tumor treating fields therapy to the tumor of the subject, the tumor treating fields therapy region comprising the first radiation therapy region;
identify a first dosage for the tumor treating fields therapy for the first radiation therapy region, wherein combining the first dosage of radiation therapy and the first dosage for the tumor treating fields therapy in the first radiation therapy region meets or exceed a therapeutic threshold for treating the tumor in the first radiation therapy region; and
select one or more transducer layouts for delivering tumor treating fields to the subject based on the first dosage for the tumor treating fields therapy for the first radiation therapy region.

20. A non-transitory processor readable medium containing a set of instructions thereon that when executed by a processor cause the processor to:
obtain a three-dimensional model of the subject, the model comprising voxels;
identify a radiation therapy region in the three-dimensional model of the subject for delivering radiation therapy to a tumor of the subject, the radiation therapy region comprising a first radiation therapy region to receive a first dosage of radiation therapy and a second radiation therapy region to receive a second dosage of radiation therapy, the first dosage of radiation therapy being less than the second dosage of radiation therapy;
identify a tumor treating fields therapy region in the three-dimensional model of the subject for delivering tumor treating fields therapy to the tumor of the subject, the tumor treating fields therapy region comprising the first radiation therapy region;
identify a first dosage for the tumor treating fields therapy for the first radiation therapy region based on the first dosage of radiation therapy for the first radiation therapy region; and
select one or more transducer layouts for delivering tumor treating fields to the subject based on the first dosage for the tumor treating fields therapy for the second radiation therapy region.
